(19) United States
(12) Patent Application Publication
(10) Pub. No.: US 2010/0179188 A1
(43) Pub. Date: Jul. 15, 2010

(54) NITROXIDE RADICAL AS TREATMENT FOR NEURODEGENERATION

(75) Inventors: Tracey A. Rouault, Bethesda, MD (US); James B. Mitchell, Damascus, MD (US); Murali Krishna Cherukuri, Gaithersburg, MD (US); Manik Ghosh, North Potomac, MD (US)

Correspondence Address: LEYDIG, VOIT & MAYER, LTD., TWO PRUDENTIAL PLAZA, SUITE 4900, 180 NORTH STETSON AVENUE, CHICAGO, IL, 60601-6731, US

(73) Assignee: Office of Technology Transfer, NIH, Bethesda, MD (US)

(21) Appl. No.: 12/530,593

(22) PCT Filed: Mar. 10, 2008
(86) PCT No.: PCT/US08/56429

§ 371 Date: Nov. 4, 2009

Related U.S. Application Data
(60) Provisional application No. 60/894,134, filed on Mar. 9, 2007.

(51) Int. Cl. A61K 31/45; A61P 25/16; A61P 25/28

(52) U.S. Cl. 514/315

(57) ABSTRACT

A method of treating or preventing neurodegeneration in a mammal comprising administering to the mammal an effective amount of a stable nitroxide radical, such as Tempo1, as well as related methods.

### CROSS-REFERENCE TO RELATED APPLICATIONS

This patent application claims the benefit of U.S. Provisional Patent Application No. 60/894,134, filed Mar. 9, 2007, which is incorporated by reference.

### BACKGROUND OF THE INVENTION

Neurodegenerative disease affects millions of people worldwide. It is believed that deficiency in the amount of bioavailable iron in the brain contributes to neurodegeneration. In particular, it is believed that Iron Regulatory Proteins, particularly IRP1 and IRP2 are involved in the impaired iron homeostasis observed in patients suffering from neurodegenerative diseases. IRP1 and IRP2 regulate the expression of ferritin, transferring receptor 1 (TfR1), and other genes by binding to iron-responsive elements within transcripts. Animals that lack IRP2 develop anemia and adult-onset progressive neurodegeneration due to decreased TfR1 expression and resulting functional iron deficiency in developing erythroid cells and in the central nervous system (CNS). Animals that lack IRP1 have only subtle perturbation of iron metabolism because IRP2 compensates for the loss of IRP1. In animals that lack IRP2, however, ferrite levels increase and TfR levels decrease in most tissues, resulting in a deficiency in the amount of iron that is available for use.

Accordingly, there is a desire for compounds that can be used to slow, treat, or prevent neurodegeneration, or otherwise treat or prevent abnormalities in iron metabolism, IRP function, or TfR1 expression.

### BRIEF SUMMARY OF THE INVENTION

The invention provides a method of treating or preventing neurodegeneration in a mammal afflicted with a neurodegenerative disease comprising administering to the mammal an amount of a stable nitroxide radical sufficient to treat or prevent neurodegeneration.

The invention also provides a method of increasing the amount of bioavailable iron in the central nervous system (CNS) of a mammal with a CNS iron deficiency comprising administering to the mammal a stable nitroxide radical in an amount sufficient to increase the amount of bioavailable iron in the central nervous system of the mammal.

The invention further provides a method of activating Iron Regulatory Protein 1 (IRP1) in a mammal comprising administering to the mammal a stable nitroxide radical in an amount sufficient to activate IRP1 in the mammal.

The invention additionally provides a method of increasing Transferrin Receptor 1 (TfR1) expression in a mammal comprising administering to the mammal a stable nitroxide radical in an amount sufficient to increase TfR1 expression.

### BRIEF DESCRIPTION OF THE SEVERAL VIEWS OF THE DRAWING(S)

The following Figures illustrate at least some embodiments of the invention:

FIGS. 1a, 1b, and 1c are graphs of hang test results for wild type (WT) and IRP2−/− mice fed a control diet or a Tempo1 supplemented diet.

FIGS. 2a and 2b are gels showing iron-responsive element (IRE) binding activity of IRP1 and protein levels of TfR1, L-ferritin (L-Ft), IRP1, and Tubulin in mouse embryonic fibroblasts. FIG. 2c presents some of the results according to the relative intensity of the gel bands.

FIG. 3a are gels showing IRE binding activity of IRP1 and protein levels of TfR1, IRP1, and Actin in the cerebellum, forebrain and brain-stem regions of IRP2−/− animals fed control (Ctrl) or Tempo1 (Tem) diets. FIG. 3b presents some of the results according to the relative intensity of the gel bands.

FIG. 4a is a gel showing ferritin and actin protein levels cerebellar lysates from wild type and IRP2−/− mice fed control or Tempo1 diets. FIG. 4b presents some of the results according to the relative intensity of the gel bands.

FIGS. 4c-4f are photographs showing relative ferritin and ferric iron levels in various regions of the brains of wild type and IRP2−/− mice though immunohistochemistry and Perls' DAB staining.

FIG. 5a depicts a gel showing the cytosolic and mitochondrial aconitase activity in mouse embryonic fibroblast lysates from wild type, IRP2−/−, and IRP1−/− mice.

FIG. 5b depicts a gel showing IRP1 and IRP2 levels in mouse embryonic fibroblasts after treatment with Tempo1 or iron-cheltor deferiprone (DFO).

FIG. 5c depicts a gel showing cytosolic and mitochondrial aconitase activity, as well as IRP1 and IRP2 protein levels, of mouse embryonic fibroblastst after treatment with Tempo1 or DFO.

FIGS. 6a and 6b depict gels showing IRE-binding activity of purified holo-IRP1 by IRE gel shift assay using treatment samples incubated with β-mercaptoethanol.

FIGS. 6c and 6d are graphs of aconitase activity over time measured by a coupled solution assay.

FIGS. 7a and 7b depict gels showing IRP1, IRP2, and Actin protein levels in erythroblast cells and forebrain lysates.

FIG. 7c is a graph of hang-test results for wild type, IRP2−/−, and IRP1+/−IRP2−/− mice fed control or Tempo1 supplemented diets.

FIG. 7d shows a proposed mechanism by which Tempo1 can directly destabilize the iron-sulfur cluster of IRP1 to recruit IRE binding activity.

### DETAILED DESCRIPTION OF THE INVENTION

Stable nitroxide radicals include compounds having the general formula R₂NO. Any suitable nitroxide radical can be used in accordance with the invention, provided it is physiologically acceptable in the mammal with which the invention is to be used. If administered systemically, the selected nitroxide radical desirably can penetrate the blood brain barrier of the chosen mammal. Preferred stable nitroxide radicals for use in the methods of the present invention include Tempo1 or a hydroxylamine analogue thereof, such as Tempo1-H. Tempo1 is a free radical scavenger, a recycling antioxidant, and it can be added to animal feed and is absorbed across the blood-brain barrier. Stable nitroxide radicals are good scavengers for free radicals, wherein an electron of the stable nitroxide forms a stable electron pair with the electron of a reactive radical.

Other stable nitroxide radicals suitable for use in accordance with the invention are known in the art. Generally, stable nitroxide radicals useful in the invention have the general formula R₂NO wherein the two R groups can be the same or different. Typically, each R group is independently selected from the group consisting of H, hydroxyl, halogen, CN, NO₂, sulfonamide, C₁-C₈ alkyl, C₃-C₆ cycloalkyl, C₁-C₆ alkoxy, C₁-C₆ haloalkoxy, C₁-C₄ haloalkyl, C₂-C₈ alkenyl, C₂-C₈ alkynyl, amino, C₁-C₄ dialkyl amino, C₁-C₄ alkylamino, C₁-C₆ cycloalkyl amino, morpholine, heteroaryl (including without limitation thienyl, pyridyl and pyrimidinyl), arylamino, arylalkylamino, phenyl, C(O)R′, NR′(COR″), NR′SO₂R″ and NR′(CONR″R′″), wherein in R′, R″ and R′″ are independently H, C₁-C₆ alkyl, phenyl, or substituted phenyl, and wherein the C₁-C₈ alkyl is optionally substituted with one or more members selected from the group consisting of C₁-C₄ alkoxy, haloalkyl, C₁-C₆ dialkyl amino, C₁-C₆ alkylamino, cycloalkylamino, and morpholine, and the phenyl is optionally substituted with one or more members selected from the group consisting of halogen, NO₂, CN, C₁-C₄ alkyl, C₁-C₄ haloalkyl, and C₁-C₄ alkoxy, or R₃ and R₄ taken together with the carbon to which they are attached, form a ring.

As used herein, unless otherwise specified, the term “alkyl” means a saturated straight chain or branched non-cyclic hydrocarbon having an indicated number of carbon atoms (e.g., C₁-C₂₀, C₁-C₁₀, C₁-C₄, etc.). Representative saturated straight chain alkyls include -methyl, -ethyl, -n-propyl, -n-butyl, -n-pentyl, -n-hexyl, -n-heptyl, -n-octyl, -n-nonyl and -n-decyl; while representative saturated branched alkyls include -isopropyl, -sec-butyl, -isobutyl, -tert-butyl, -isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like. An alkyl group can be unsubstituted or substituted.

As used herein, unless otherwise specified, the term “cycloalkyl” means a monocyclic or polycyclic saturated ring comprising carbon and hydrogen atoms and having no carbon-carbon multiple bonds. Examples of cycloalkyl groups include, but are not limited to cyclopropyl, cyclobutyl, cyclopentyl, cyclohexyl, and cycloheptyl, and saturated cyclic and bicyclic terpenes. A cycloalkyl group can be unsubstituted or substituted. Preferably, the cycloalkyl group is a monocyclic ring or bicyclic ring.

As used herein, unless otherwise specified, the term “alkenyl group” means a straight chain or branched non-cyclic hydrocarbon having an indicated number of carbon atoms (e.g., C₂-C₂₀, C₂-C₁₀, C₂-C₄, etc.). Representative straight chain and branched alkenyls include -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexenyl, -2-hexenyl, -3-hexenyl, -1 -heptenyl, -2-heptenyl, -3-heptenyl, - 1-octenyl, -2-octenyl, -3-octenyl, -1-nonenyl, -2-nonenyl, -3-nonenyl, -1-decenyl, -2-decenyl, -3-decenyl and the like. The double bond of an alkenyl group can be unconjugated or conjugated to another unsaturated group. An alkenyl group can be unsubstituted or substituted.

As used herein, unless otherwise specified the term “alkynyl group” means a straight chain or branched non-cyclic hydrocarbon having an indicated number of carbon atoms (e.g., C₂-C₂₀, C₂-C₁₀, C₂-C₆, etc.), and including at least one carbon-carbon triple bond. Representative straight chain and branched alkynyls include -acetylenyl, -propynyl, -1-butynyl, -2-butynyl, -1-pentynyl, -2-pentynyl, -3-methyl-1-butynyl, -4-pentynyl, -1-hexynyl, -2-hexynyl, -5-hexynyl, -1-heptynyl, -2-heptynyl, -6-heptynyl, -1-octynyl, -2-octynyl, -7- octynyl, -1-nonynyl, -2-nonynyl, -8-nonynyl, -1-decynyl, -2-decynyl, -9-decynyl, and the like. The triple bond of an alkynyl group can be unconjugated or conjugated to another unsaturated group. An alkynyl group can be unsubstituted or substituted.

As used herein, unless otherwise specified, the term “halogen” or “halo” means fluorine, chlorine, bromine, or iodine. Furthermore, unless otherwise specified, the term “haloalkyl” means an alkyl substituted with one or more halogens, wherein alkyl and halogen are defined as above.

As used herein, unless otherwise specified, the term “alkoxy” means —O-(alkyl), wherein alkyl is defined above. Furthermore, as used herein, the term “haloalkoxy” means an alkoxy substituted with one or more halogens, wherein alkoxy and halogen are defined as above.

As used herein, unless otherwise specified, the term “heteroaryl” means a carbocyclic aromatic ring containing from 5 to 14 ring atoms comprising at least one heteroatom, preferably 1 to 3 heteroatoms, independently selected from nitrogen, oxygen, or sulfur. Heteroaryl ring structures include compounds having one or more ring structures, such as mono-, bi-, or tricyclic compounds, as well as fused heterocyclic moities. Representative heteroaryls are triazolyl, tetrazolyl, oxadiazolyl, pyridyl, furanyl, benzofuranyl, thiophenyl, thiazolyl, benzothiophenyl, benzoisoxazolyl, benzoisothiazolyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl, quinazolinyl, benzoquinazolinyl, acridinyl, pyrimidyl, oxazolyl, benzo[1,3]dioxole, and 2,3-dihydro-benzo[1,4]dioxine. A heteroaryl group can be unsubstituted or substituted.

As used herein, unless otherwise specified, the term “alkylamino” means —NH(alkyl) or —N(alkyl)(alkyl), wherein alkyl is defined above. As used herein, unless otherwise specified, the term “aminoalkyl” means -(alkyl)-NH₂, wherein alkyl is defined above.

As used herein, unless otherwise specified, the term “substituted” means a group substituted by one to four or more substituents, such as, alkyl, alkenyl, alkynyl, cycloalkyl, aroyl, halo, haloalkyl (e.g., trifluoromethyl), haloalkoxy (e.g., trifluoromethoxy), hydroxy, alkoxy, alkylthioether, cycloalkyloxy, heterocylooxy, oxo, alkanoyl, aryl, arylalkyl, alkylaryl, heteroaryl, heteroarylalkyl, alkylheteroaryl, heterocyclo, aryloxy, alkanoyloxy, amino, alkylamino, arylamino, arylalkylamino, cycloalkylamino, heterocycloamino, mono- and di-substituted amino (in which the two substituents on the amino group are selected from alkyl, aryl or arylalkyl), alkanoylamino, aroylamino, aralkanoylamino, substituted alkanoylamino, substituted arylamino, substituted aralkanoylamino, thiol, alkylthio, arylthio, arylalkylthio, cycloalkylthio, heterocyclothio, alkylthiono, arylthiono, arylalkylthiono, alkylsulfonyl, arylsulfonyl, arylalkylsulfonyl, sulfonamido (e.g., SO₂NH₂), substituted sulfonamido, nitro, cyano, carboxy, carbamyl (e.g., CONH₂), substituted carbamyl (e.g., CONH-alkyl, CONH-aryl, CONH-arylalkyl or instances where there are two substituents on the nitrogen selected from alkyl or arylalkyl), alkoxycarbonyl, aryl, substituted aryl, guanidino, substituted or unsubstituted heterocycloalkyl, substituted or unsubstituted heteroaryl (such as, indolyl, imidazolyl, furyl, thienyl, thiazolyl, pyrrolidyl, pyridyl, pyrimidyl and the like).

Whenever a range of the number of atoms in a structure is indicated (e.g., a C₁-C₈, C₁-C₆, C₁-C₄, or C₁-C₃ alkyl, haloalkyl, alkylamino, alkenyl, etc.), it is specifically contemplated that any sub-range or individual number of carbon atoms falling within the indicated range also can be used. Thus, for instance, the recitation of a range of 1-8 carbon atoms (e.g., C₁-C₈), 1-6 carbon atoms (e.g., C₁-C₆), 1-4 carbon atoms (e.g., C₁-C₄), 1-3 carbon atoms (e.g., C₁-C₃), or 2-8 carbon atoms (e.g., C₂-C₈) as used with respect to any chemical group (e.g., alkyl, haloalkyl, alkylamino, alkenyl, etc.) referenced herein encompasses and specifically describes 1, 2, 3, 4, 5, 6, 7, or 8 carbon atoms, as appropriate, as well as any sub-range thereof (e.g., 1-2 carbon atoms, 1-3 carbon atoms, 1-4 carbon atoms, 1-5 carbon atoms, 1-6 carbon atoms, 1-7 carbon atoms, 1-8 carbon atoms, 2-3 carbon atoms, 2-4 carbon atoms, 2-5 carbon atoms, 2-6 carbon atoms, 2-7 carbon atoms, 2-8 carbon atoms, 3-4 carbon atoms, 3-5 carbon atoms, 3-6 carbon atoms, 3-7 carbon atoms, 3-8 carbon atoms, 4-5 carbon atoms, 4-6 carbon atoms, 4-7 carbon atoms, 4-8 carbon atoms, 5-6 carbon atoms, 5-7 carbon atoms, 5-8 carbon atoms, 6-7 carbon atoms, or 6-8 carbon atoms, as appropriate).

Without wishing to be bound by any particular theory, it is believed that the administration of a stable nitroxide radical to a mammal increases the activity of IRP1 by removing an inhibitory iron-sulfur cluster from a site that otherwise can bind mRNA and regulate the expression of TfR1 and ferritin. Such regulation results in increased iron uptake and decreased sequestration of iron into inaccessible proteins. Thus, it is believed that the stable nitroxide allows IRP1 to supplement for IRP2 deficiency. Accordingly, the methods of the invention are believed to be especially useful for administration to a mammal deficient in Iron Regulatory Protein 2 (IRP2) function, and, thus, for the treatment of any disease associated with IRP2 deficiency. Also, due to its ability to upregulate TfR1 expression, directly or indirectly, the methods of the present invention are believed to be useful for administration to a mammal that under-expresses TfR1, and, thus, for the treatment of any disease associated with TfR1 underexpression. In the context of the invention, underexpression is intended to encompass reduced activity of a protein for any reason including, without limitation, reduced protein levels, the presence of other factors that inhibit the function of the normal protein, or mutations in the protein that affect its function.

The invention therefore provides a method of increasing the amount of bioavailable iron in the central nervous system (CNS) of a mammal with a CNS iron deficiency comprising administering to the mammal a stable nitroxide radical in an amount sufficient to increase the amount of bioavailable iron in the central nervous system of the mammal. By “increase in the amount of bioavailable iron” is meant an increase in bioavailable iron in the mammal after administration of the stable nitroxide radical as compared to the amount of bioavailable iron in the mammal prior to administration (or in the absence) of the stable nitroxide radical. Preferably, the amount of bioavailable iron is increased by about 10% or more, 15% or more, 20% or more, 25% or more, 50% or more, or 100% or more. “Bioavailable” means available or accessible for use by the cells of the mammal. Methods for measuring and comparing the amounts of bioavailable iron are known in the art.

The invention further provides a method of activating Iron Regulatory Protein 1 (IRP1) in a mammal comprising administering to the mammal a stable nitroxide radical in an amount sufficient to activate IRP1 in the mammal. IRP1 is activated if the activity of IRP1 in a mammal, or a biological sample isolated from a mammal, is greater after administration of the stable nitroxide radical than the activity of IRP1 in the mammal or biological sample obtained from the mammal prior to (or in the absence of) administration of the stable nitroxide radical. Preferably, IRP1 activity is increased by at least about 10% or more, 15% or more, 20% or more, 25% or more, 50% or more, 100% or more, or even 500% or more. Methods for measuring and comparing the activity of IRP1 are known in the art.

The invention additionally provides a method of increasing Transferrin Receptor 1 (TfR1) expression in a mammal comprising administering to the mammal a stable nitroxide radical in an amount sufficient to increase TfR1 expression. The increase in TfR1 expression can be any increase in TfR1 expression in the mammal or biological sample from the mammal after administration of the stable nitroxide radical as compared to the TfR1 expression in the mammal or biological sample from the mammal prior to administration of the stable nitroxide radical (or in the absence of the nitroxide radical). An increase in TfR1 expression can include an increase in the relative amount of TfR1 present, or an increase in the biological activity of TfR1, for example, without increasing the amount of TfR1. Preferably, TfR1 expression is increased by about 10% or more, 15% or more, 20% or more, 25% or more, 50% or more, 100% or more, or even 500% or more. Methods for measuring and comparing the expression of TfR1 are known in the art.

The methods of the invention can be used for any purpose, such as for the research, diagnosis, prevention, or treatment of disease relating abnormal (e.g., lower than normal) levels of bioavailable iron, abnormal (e.g., lower than normal) IRP1 or IRP2 activity levels, or abnormal (e.g., lower than normal) levels of TfR1 expression. Such conditions can be associated with neurodegeneration. Thus, any of the foregoing methods can be used in conjunction with the research, diagnosis, prevention, or treatment of a neurodegenerative disease.

The invention therefore provides, in another aspect, a method of treating or preventing neurodegeneration in a mammal afflicted with a neurodegenerative disease comprising administering to the mammal an amount of a stable nitroxide radical sufficient to treat or prevent neurodegeneration. Treating or preventing neurodegeneration in a mammal includes treating or preventing any one or more symptoms of neurodegeneration. Such symptoms are known in the art, some of which are illustrated by the Examples.

Any of the methods of the invention can be used in conjunction with a mammal afflicted with a neurodegenerative disease or neurodegenerative condition, especially a neurodegenerative disease or condition characterized by abnormal iron metabolism (e.g., abnormal accumulations of ferric iron in the CNS), a deficiency in IRP function (e.g., IRP2 mutation or deletion), or underexpression of TfR1. Humans with IRP2 deficiency, partial or complete, would be expected to have adult-onset neurodegenerative disease, possibly associated with a mild microcytic anemia, elevated serum ferritin and elevated levels of protoporphyrin IX in red cells. By way of illustration, such diseases or conditions may include Parkinson's Disease, Alzheimer's Disease, Hallevorden-Spatz, aceruloplasminemia, refractory anemia, Friedreich ataxia, erythropoietic protoporphyria, or adult-onset neurodegeneration. Of course, the methods of the invention also can be used in conjunction with a mammal with a deficiency in IRP function or underexpression of TfR1, which has not shown signs of neurodegeneration. Such application of the methods of the invention would be useful, for example, in restoring IRP function, TfR1 expression, and/or iron metabolism, as well as, perhaps, preventing or delaying the onset of neurodegeneration.

Any of the methods of the invention can be further implemented in conjunction with the step of administering to the mammal an iron supplement or effectively high iron diet. In IRP2 deficient mammals, a functional iron deficiency can be supplemented with appropriate iron compounds known to those of skill in the art in order to further augment the benefits obtained through administration of stable nitroxide radicals, such as Tempo1. Such a high iron diet or other iron supplement can be administered by any suitable method such as those discussed below with reference to stable nitroxide radical administration.

The stable nitroxide radical can be administered by any suitable method. For example, the stable nitroxide radical can be administered by oral, aerosol, parenteral, subcutaneous, intravenous, intramuscular, interperitoneal, or intraarterial administration. Suitable formulations of Tempo1 for use in conjunction with the method of the invention are known in the art.

The nitroxide radical can be formed as a composition, such as a pharmaceutical composition, comprising a compound and a carrier, especially a pharmaceutically acceptable carrier. The pharmaceutical composition can comprise two or more different nitroxide radicals. Alternatively, or in addition, the pharmaceutical composition can comprise one or more nitroxide radicals in combination with other pharmaceutically active agents or drugs, including drugs known to be useful for the treatment or prevention of any of the aforementioned diseases or symptoms associated therewith (e.g., levodopa, carbidopa, dopamine agonists (Parlodel, Permax, Requip, Mirapex, Symmetrel), anticholinergics (Artane, Cogentin), Eldepryl, COMT Inhibitors (Tasmar, Comtan), non-steroidal anti-inflammatory drugs (NSAIDs), GSK-3 inhibitors, etc.). Co-administration or sequential administration of the nitroxide radical with such other drugs also can be used.

The composition further comprises a carrier. The carrier can be any suitable carrier. Preferably, the carrier is a pharmaceutically acceptable carrier. With respect to pharmaceutical compositions, the carrier can be any of those conventionally used and is limited only by physio-chemical considerations, such as solubility and lack of reactivity with the active compound(s), and by the route of administration. It will be appreciated by one of skill in the art that, in addition to the following described pharmaceutical composition, the compounds and inhibitors of the present inventive methods can be formulated as inclusion complexes, such as cyclodextrin inclusion complexes, or liposomes.

The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the active agent(s) and one which has no detrimental side effects or toxicity under the conditions of use.

The choice of carrier will be detenained in part by the particular nitroxide radical and other active agents or drugs used, as well as by the particular method used to administer the compound and/or inhibitor. Accordingly, there are a variety of suitable formulations of the pharmaceutical composition of the present inventive methods. The following formulations for oral, aerosol, parenteral, subcutaneous, intravenous, intramuscular, interperitoneal, rectal, and vaginal administration are exemplary and are in no way limiting. One skilled in the art will appreciate that these routes of administering the nitroxide radical are known, and, although more than one route can be used to administer a particular compound, a particular route can provide a more immediate and more effective response than another route.

Injectable formulations are among those formulations that are useful in accordance with the present invention. The requirements for effective pharmaceutical carriers for injectable compositions are well-known to those of ordinary skill in the art (See, e.g., Pharmaceutics and Pharmacy Practice, J. B. Lippincott Company, Philadelphia, Pa., Banker and Chalmers, eds., pages 238 250 (1982), and ASHP Handbook on Injectable Drugs, Toissel, 4th ed., pages 622 630 (1986)).

Topical formulations are well known to those of skill in the art. Such formulations are particularly suitable in the context of the present invention for application to the skin.

Formulations suitable for oral administration can consist of (a) liquid solutions, such as an effective amount of the inhibitor dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant. Capsule forms can be of the ordinary hard or soft shelled gelatin type containing, for example, surfactants, lubricants, and inert fillers, such as lactose, sucrose, calcium phosphate, and corn starch. Tablet fauns can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

The pharmaceutical composition can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non pressured preparations, such as in a nebulizer or an atomizer. Such spray formulations also may be used to spray mucosa.

Formulations suitable for parenteral administration include aqueous and non aqueous, isotonic sterile injection solutions, which can contain anti oxidants, buffers, bacteriostats, and solutes that render the formulation isotonic with the blood of the intended recipient, and aqueous and non aqueous sterile suspensions that can include suspending agents, solubilizers, thickening agents, stabilizers, and preservatives. The nitroxide radical can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, dimethylsulfoxide, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive; petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-b-aminopropionates, and 2-alkyl-imidazoline quaternary ammonium salts, and (e) mixtures thereof.

The parenteral formulations will typically contain from about 0.5% to about 25% by weight of the active ingredient in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Additionally, the pharmaceutical composition can be made into suppositories by mixing with a variety of bases, such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate.

One of ordinary skill in the art will readily appreciate that nitroxide radicals can be modified in any number of ways to increase the therapeutic efficacy of the compound. For instance, the nitroxide radical could be conjugated either directly or indirectly through a linker to a targeting moiety. The practice of conjugating compounds to targeting moieties is known in the art. The term “targeting moiety” as used herein, refers to any molecule or agent that specifically recognizes and binds to a cell-surface receptor, such that the targeting moiety directs the delivery of the compound or inhibitor to a population of cells on which surface the receptor is expressed. Targeting moieties include, but are not limited to, antibodies, or fragments thereof, peptides, hormones, growth factors, cytokines, and any other naturally- or non-naturally-existing ligands, which bind to cell surface receptors. The term “linker” as used herein, refers to any agent or molecule that bridges the compound to the targeting moiety. One of ordinary skill in the art recognizes that sites on the compounds which are not necessary for the function of the compound or inhibitor are ideal sites for attaching a linker and/or a targeting moiety, provided that the linker and/or targeting moiety, once attached to the compound, do(es) not interfere with its function.

Alternatively, the nitroxide radical can be modified into a depot form, such that the manner in which the nitroxide radical is released into the body to which it is administered is controlled with respect to time and location within the body (see, e.g., U.S. Pat. No. 4,450,150). Depot forms can be, for example, an implantable composition comprising the nitroxide radical and a porous material, such as a polymer, wherein the nitroxide radical is encapsulated by or diffused throughout the porous material. The depot is then implanted into the desired location within the body and the active ingredient is released from the implant at a predetermined rate by diffusing through the porous material.

In some contexts, the nitroxide radical can be advantageously administered via an implanted pump that allows intrathecal delivery. Such a delivery method is especially useful for delivery of drugs to the CNS when the drugs administered do not otherwise sufficiently penetrate the blood-brain barrier.

The nitroxide radicals described herein can be administered to a cell in vitro to achieve any of the effects hereinbefore mentioned with respect to the administration of a nitroxide radical to a mammal. As used herein, the term “in vitro” means that the cell is not in a living organism. The nitroxide radical also can be administered to a cell in vivo. As used herein, the term “in vivo” means that the cell is a part of a living organism or is the living organism. Furthermore, the nitroxide radical can be administered to a host in vivo or ex vivo. The term “ex vivo” as used herein refers to the administration of a compound to a cell or a population of cells in vitro, followed by administration of the cell or population of cells to a host.

Furthermore, the nitroxide radical can be administered alone, or in conjunction with of an agent that enhances the efficacy of the nitroxide radical. Such agents can include, for instance, any of the other active agents described herein with respect to the pharmaceutical composition, which agents can be administered in a composition separate from the composition comprising the nitroxide radical.

The amount or dose of the nitroxide radical should be sufficient to effect a therapeutic or prophylactic response in the host over a reasonable time frame. The appropriate dose will depend upon the nature and severity of the disease or affliction to be treated or prevented, as well as by other factors. For instance, the dose also will be determined by the existence, nature and extent of any adverse side effects that might accompany the administration of a particular compound. Ultimately, the attending physician will decide the dosage with which to treat each individual patient, taking into consideration a variety of factors, such as age, body weight, general health, diet, sex, inhibitor to be administered, route of administration, and the severity of the condition being treated. Typically doses might be, for example, 0.1 mg to 1 g daily, such as 5 mg to 500 mg daily.

The methods of the invention can be used in conjunction with any type of mammal. Mammals as discussed herein include, but are not limited to, the order Rodentia, such as mice, and the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human. Furthermore, the mammal can be the unborn offspring of any of the forgoing hosts, especially mammals or humans, in which case administration of compounds can be performed in utero.

The methods can be used for any purpose, including but not limited to the research, treatment, or prevention of any of the diseases or conditions discussed herein, other diseases or conditions.

The following examples further illustrate the invention but, of course, should not be construed as in any way limiting its scope.

### Examples

Background on Experimental Model: In the following examples, IRP2−/− mice are used to model neurodegerative disease. The neurodegeneration of IRP2−/− animals is characterized by progressive loss of motor capabilities, for example as measured in mice by performance on hang-tests, rotarod rotating drum treadmill testing, balance beams, climbing pole tests, allelerods, footprints, decreased grooming activities and gait abnormalities in adult animals. The neurodegeneration of IRP2−/− animals progresses slowly as animals age.

It is believed that iron regulatory proteins (IRPs) regulate cellular iron homeostasis by binding to RNA stem-loops known as iron-responsive elements (IREs) found within transcripts that encode iron metabolism proteins. For instance, IRP binding to the IRE at the 5′end of ferritin H or L transcripts represses ferritin translation, whereas IRP binding to IREs in the 3′UTR of TfR1, and one isoform of the metal transporter, DMT1, stabilizes the mRNA. Ferritin levels are abnormally high in most tissues of IRP2−/− animals, whereas TfR1 levels are abnormally low.

Mice that lack IRP2 develop microcytic anemia and neurodegeneration associated with functional cellular iron depletion caused by low TfR1 and high ferritin expression. IRP1−/− animals do not significantly misregulate iron metabolism, partly because IRP1 is an iron-sulfur protein that functions mainly as a cytosolic aconitase in mammalian tissues, and IRP2 activity increases to compensate for loss of the IRE binding form of IRP1. Thus far, no phenotypes attributable to loss of cytosolic aconitase have been identified.

Notably, IRP2−/− animals that also lack one IRP1 allele (IRP1−/+) show greater misregulation of IRP target transcripts along with increased severity of anemia and neurodegeneration, indicating that the small fraction of IRP1 that has IRE binding activity contributes to regulation of intracellular iron metabolism. Consistent with the notion that the IRE binding activity of IRP1 is important in iron homeostasis, animals that lack both alleles of IRP1 in addition to IRP2 (IRP1−/− IRP2−/−) do not survive beyond the blastocyst stage of development.

IRP2−/− mice develop a progressive neurodegenerative disease that can be observed by Ferric staining of white matter from mice brains. In enhanced Penls' DAB stains and Aminocupric silver stains, axonal iron can be observed accumulating co-locally with axonal degeneration. Axonal inflammation also appears widespread in IRP2−/− mice, in areas such as the ventral spinal nerve root and in the cervical spinal cord. Early signs of degeneration of the neuronal cell body include darkening of nucleoplasm, loss of nuclear membrance integrity, and blebbing of plasma membrane, which can be observed by comparative analysis of superior colliculus samples against control WT mice. Vacuoles that conform to the size and shape of neurons are found throughout affected regions of the brain. Numerous vacuoles appear to be present with the substantia nigra of IRP2−/− mice. Axonal degeneration appears, at least in part, due to iron toxicity from ferritin turnover and due to iron sequestration by ferritin, coupled with low TfR expression that leads to functional iron deficiency and mitochondrial insufficiency. Maldistribution of iron in apparent “iron overload” can be associated with functional iron deficiency, whereby ferritin sequesters iron at the expense of other iron proteins and where IRP2−/− mice have iron-deficiency anemia because deficient TfR expression on developing erythrocytes. Iron-insufficiency anemia of IRP 2−/− mice is observed with decreased hemocrit levels compared to WT and elevated levels of free and zinc protoporphyrins. Moreover, the bone marrow of IRP2−/− animals appears to be completely iron deficient when examined as Perls' iron stain.

Additionally, Western blot images at about 97 kD indicate that transferring receptor levels decrease in erythroid hematopoietic cells from IRP2−/− amd IRP1+/− IRP 2−/− mice, while ferritin L and H Teves increase by images at about 36 kD and 22 kD. Translation of eALAS increases relative to WT controls by images at about 64 kD and 51 kD. Over-expression of eALAS leads to increased protoporphyrin IX synthesis, and iron deficiency appears to prevent heme formation. IRP2 deficiency can cause erythropoietic protoporphryia (EPP). Non-heme brain iron content has been observed to decrease in IRP2−/− animals, for example in mice WT at about 78.9+/− 9 ug/gram dry weight goes to about 62.2+/− 12 in IRP2−/− mice with a P<0.01. But even with similar or slightly decreased total non-heme brain iron, overall ferric iron appears relatively increased in IRP2−/− brains (by about over a factor of four), while bioavailable ferrous iron is relatively decreased with respect to WT (by about over a factor of four).

TfR appears important in brain iron uptake and iron-sulfur clusters appear important in mitochondrial respiratory complexes. Mitochondria are required for axonal maintenance. Loss of axonal integrity appears widespread in IRP2−/− mice. Based on genotyping analysis, NF-κB appears to be an important gene in regulation of neuronal activity-dependant transcription and behavior of axons. Retrograde transport enables NF-κB to transcriptionally activate target genes involved in neuronal well-being. Thus, without wishing to be bound by any particular theory, it is believed that the pathogenesis of neurodegeneration in IRP2−/− mice results from iron deficiency that compromises mitochondrial function, decreased ATP production leads to axonal swelling and decreased axonal transport, and the inability of NF-κB to move to nucleus results in decreased expression of numerous proteins important in neuronal maintenance and well-being. By administering a stable nitroxide radical, such as Tempo1, for IRP2 deficient patients, a useful treatment to prevent neurodegeneration is obtained.

Methods:

Mice: IRP2−/− mice were generated, propagated by breeding and genotyped as described in LaVaute et al., Nature Genetics, 27, 209-214 (2001). Mice used in this study have a 129S4/SvJae X C57B1/6 mixed background (specific proportions of each strain are not known). In experiments with mice of same genotype but on different diets siblings were used to minimize phenotypic variation due to differences in genetic background. Mice of different genotypes and on different diets were age and sex matched. All protocols were approved by the National Institute of Child Health and Human Development Animal Care and Use Committee, and met US National Institutes of Health guidelines for the humane care of animals.

Diet: The mice were weaned 3-4 weeks after their birth. Immediately after weaning, mice were maintained on either a Tempo1-supplemented or control diet. In the Tempo1-supplemented diet, powdered Tempo1 was uniformly mixed with bacon-flavored mouse chow by a “cold press” technique (Bio-Serv, Frenchtown, N.J., USA) at a concentration of 10 mg/g of food. Bacon-flavored chow without Tempo1 was used as the control diet.

Hang-test: In the hang-test, mice were allowed to grip a wire mesh that was then inverted. The length of time that a mouse could hang on to an inverted wire mesh before falling (up to a maximum of 60 seconds) was measured and recorded.

Tissue and lysate preparation: Animals were euthanized and tissues were frozen in liquid nitrogen immediately after harvesting, and stored at −80° C. under argon. Experiments were performed on tissues that were pulverized in liquid N₂-cooled mortars in an anaerobic chamber, and then lysed in lysis buffer that was deaerated by cyclic freezethaw and air-removal with argon. Nuclei and debris were removed by centrifugation. Preparations of lysates for assays of IRP1 activity, western blotting, carbonyl assay and protein analysis were performed anaerobically.

Cells: Embryonic fibroblasts of 13-day old embryos were isolated from wild type, IRP1−/− mice and IRP2−/− mice as described in LaVaute et al. supra. Myc-tagged HEK 293 Tet-on cell line, in which IRP2 expression was inducible, was prepared and cultured as described Bourdon et al., Blood Cells Mol Dis, 31, 247-255 (2003). Erythroblasts were harvested from bone marrow and purified as described in Cooperman et al., Blood, 106, 1084-1091 (2005).

RNA mobility shift assays: Gel retardation assays were performed as described in Meyron-Holtz et al., EMBO J, 23, 386-395 (2004). Tissue lysates were prepared in an anaerobic chamber as described above in oxygen-depleted lysis buffer containing 10 mM HEPES (pH 7.2), 3 mM MgC12, 40 mM KCl, 5% glycerol, 0.2% Nonidet P-40, 5 mM DTT, 1 mM AEBSF, 10 μg/ml. Leupeptin and complete TM EDTA free protease inhibitor cocktail (Roche Applied Science, Indiana). Lysate (x μl) containing 10 μg of total protein was added to (12.5−x) μl of bandshift buffer containing 25 mM Tris-HCl (pH 7.5) and 40 mM KCl. The samples were incubated for 5 min at room temperature (RT) with 12.5 μl of a reaction cocktail containing 20% glycerol, 0.2 U/μl Super RNAsine (Ambion, Tex.), 0.6 μg/μl yeast t-RNA, 5 mM DTT and 20 nM 32P-labelled IRE from human ferritin H-chain gene in 25 mM Tris-HCl (pH 7.5) and 40 mM KCl. A measure of 20 μl of this reaction mixture was loaded into a 10% acrylamide/TBE gel, which was run at 200 V for 2.15 h, and then the gel was fixed, dried and exposed for autoradiography.

Western blotting and antibodies: Protein analysis was carried out as described in LaVaute et al., supra. Equal amounts of protein (20-40 μg/lane) were separated on 13% SDSPAGE and transferred to nitrocellulose membranes. The membrane was blocked with 5% non-fat milk, 0.1% Triton X-100 in PBS and probed at RT in the same blocking buffer. IRP1 antibody was prepared against purified hIRP1 and used at 1:5000 dilution. L-ferritin antibody was raised in rabbit from L-ferritin protein purified from mouse livers. A mouse monoclonal TFR antibody from Zymed was used at 1:2000 dilution. Monoclonal anti-α-Tubulin and anti-β-actin antibodies from SIGMA were used at 1:5000 dilution. Western blots were treated with secondary peroxidase-conjugated goat anti-rabbit IgG or sheep anti-mouse IgG antibodies from GE healthcare at 1:5000 and 1:2000 dilutions respectively. Western blots were developed using enhanced chemiluminescence (ECL kit, Pierce, Ill.).

Aconitase assay: Aconitase activity gels for human lysates were performed as described in Tong et al., infra, and aconitase activity gels for mouse lysate were performed with the following modifications. The gel was composed of a separating gel containing 6% acrylamide, 132 mM Tris base, 66 mM borate, 3.6 mM citrate, and a stacking gel containing 4% acrylamide, 66 mM Tris base, 33 mM borate, 3.6 mM citrate. The running buffer contains 25 mM Tris pH 8.3, 96 mM glycine, and 3.6 mM citrate. Electrophoresis were carried out at 170 V at 4° C. Spectral aconitase activity was measured by following the method of Fillebeen et al., infra, using cis-aconitate as the substrate.

Immunohistochemistry: Paraffin-embedded tissue sections were boiled in a micro oven for 15 min for antigen retrieval in 10 mM citrate buffer pH 6.4 after dewaxing and rehydrating. The sections were blocked in blocking buffer (Tris-buffered saline pH 7.4, 5% nomial goat serum, 0.1% Tween-20) for 30 min, then were incubated with polyclonal rabbit anti-ferritin antibody for 2 h at room temperature, the protein-antibody complex was labeled by CY3-donkey anti-rabbit antibody (Jackson ImmunoResearch Laboratories, Inc., West Grove, Pa.), nuclei were labeled by DAPI as counterstaining. The slides were observed and the pictures were recorded with Nikon Eclipse E600 fluorescence microscope.

Perl's DAB iron staining: After dewaxing and rehydrating, paraffin-embeded tissue sections were stained in prewarmed staining solution (5% potassium ferrocyanide 15.0 ml, 5% hydrochloric acid 15.0 ml, 15 sec in micro oven) for 5 min. The sections were rinsed with distilled water and Tris-buffered saline pH7.4, then were stained with DAB staining solution (20 mg DAB, 50 μl 30% H2O2, 20 ml Tris-buffered saline pH 7.4) for 30 min at room temperature. After rinsing with distilled water, the sections were mounted with Crystal Mount mounting solution (Sigma) and were observed and taken pictures with Nikon Eclipse E600 fluorescence microscope.

Statistics: We tested differences between means of hang-time by a paired Student's t-test. Results with p<0.05 were considered as statistically significant.

### Example 1

This following example demonstrates the use of Tempo1 to treat or prevent neurodegeneration in IRP2 deficient mice.

A stable nitroxide radical, Tempo1, was administered as a dietary supplement to knockout mice (IRP2−/−), and neurodegeneration was tested using the “hang test,” by which mice were allowed to grasp a wire mesh screen in an inverted position and the length of time that the mice could remain grasping the screen was measured (up to a maximum of 60 seconds). The hang test quantitatively assesses the progression of neurodegeneration in mice. Crawley et al., Brain Res. 835, 18-26 (1999).

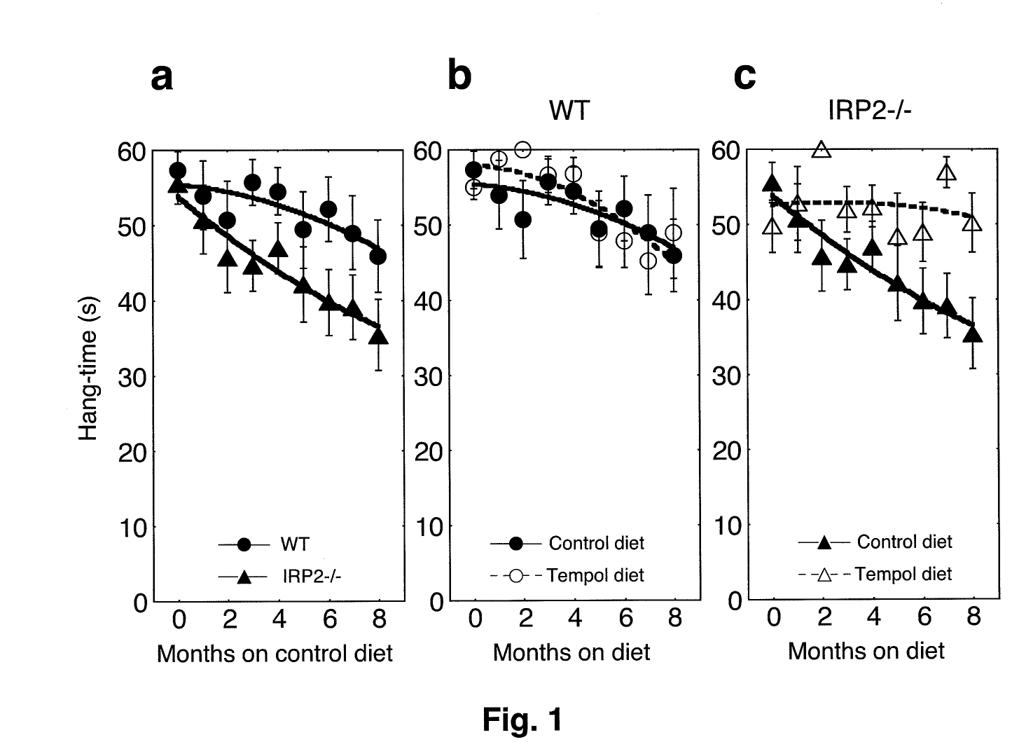

The ability of IRP2−/− mice to maintain their grip after inversion of the wire mesh diminishes progressively as animals grew older, and was significantly worse (p value=0.015) in IRP2−/− mice compared to WT mice (see FIG. 1a). The knockout mice on the Tempo1 supplemented diet exhibited substantially increased hang times as compared to knockout mice that had not been treated, indicating that IRP2−/− mice maintained on a diet supplemented with Tempo1 were significantly (p value=0.009) protected from progressive loss of neuromuscular capability (FIG. 1c). IRP2−/− mice supplemented with Tempo1 did not develop other signs of neurodegeneration such as movement disorders, tremor or abnormalities of gait and grooming.

### Example 2

The following example shows that stable nitroxide radicals provide a therapeutic benefit by way of a positive effect on activity and/or expression of iron metabolism genes.

A nitroxide radical such as tempol commonly is assumed to function as a free radical scavenger that provides therapeutic benefit by alleviating oxidative stress. However, multiple assays for oxidative stress in IRP2−/− animals, including lipid oxidation, DNA oxidation (8-hydroxyguanine assays) and protein oxidation assays, showed nothing to indicate that oxidative stress had an important role in disease progression (data not shown here).

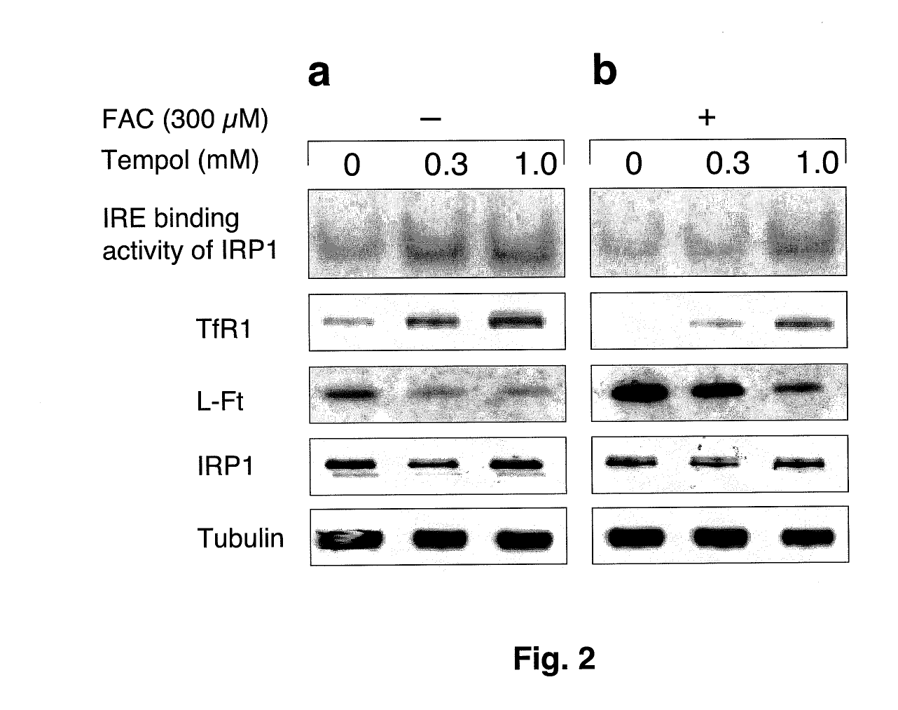

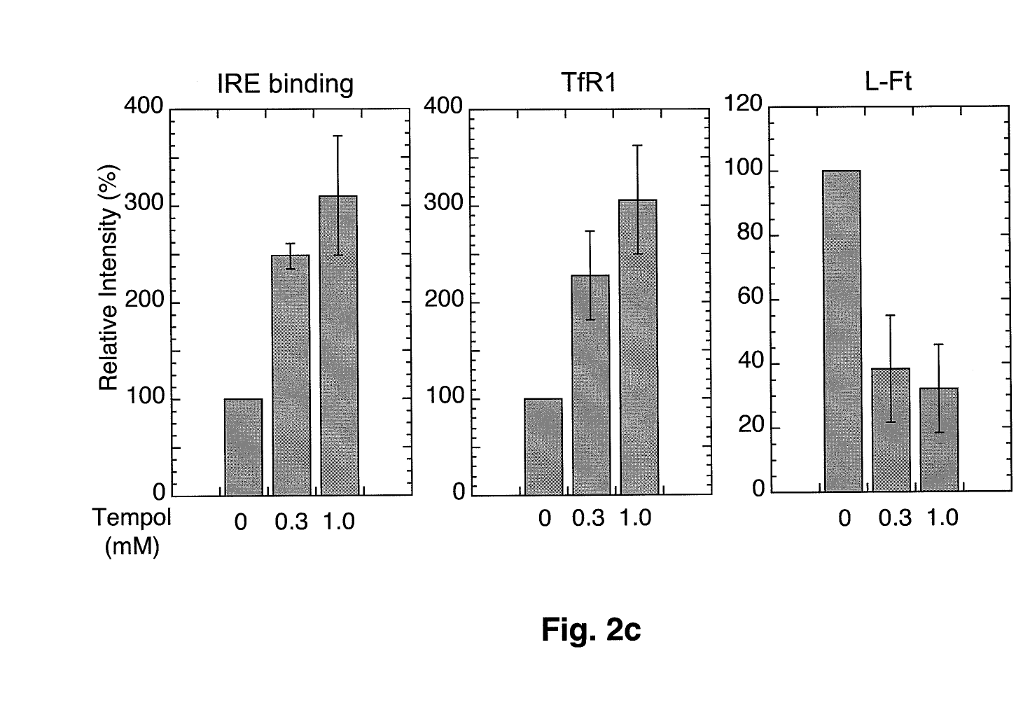

To study the effect of Tempo1 on the IRE-binding activity of IRP1, IRP2−/− embryonic fibroblasts that were maintained in culture supplemented with 0, 0.3 and 1.0 mM Tempo1, with or without ferric ammonium citrate (FAC), for 16 h. Western blots in FIGS. 2a and 2b show IRE binding activity of IRP1, as well as protein levels of TfR1, L-Ft, IRP1, and Tubulin. All IRE binding activity was attributable to IRP1 activation. The results show that TfR1 levels increased and ferritin levels decreased in Tempo1 treated cells, whereas IRP1 and tubulin levels (loading control) were unchanged. FIG. 2c shows IRE-binding activities of IRP1, and the TfR1 and L-ferritin (L-Ft) protein levels at different concentrations of Tempo1 (without added FAC) as a function of intensity as compared to the control lanes, represented here as 100%. Quantification was performed with the IQMac (IRP1 activity) or NIH Image (protein levels) program. Error bars represent the standard deviation calculated from the results of two different sets of experiments.

Taken together, these results indicate that treatment with a nitroxide radical allows cells to compensate for the loss of IRP2 by activating the latent IRE-binding activity of IRP1, thus reversing the misregulation of TfR1 and ferritin. Tempo1 and similar stable nitroxide radicals thus are an attractive neuroprotective treatment, because they activate IRP1 by a mechanism that does not cause significant free radical stress or iron depletion.

### Example 3

The following example shows the effect of a nitroxide radical on IRE binding activity in vivo.

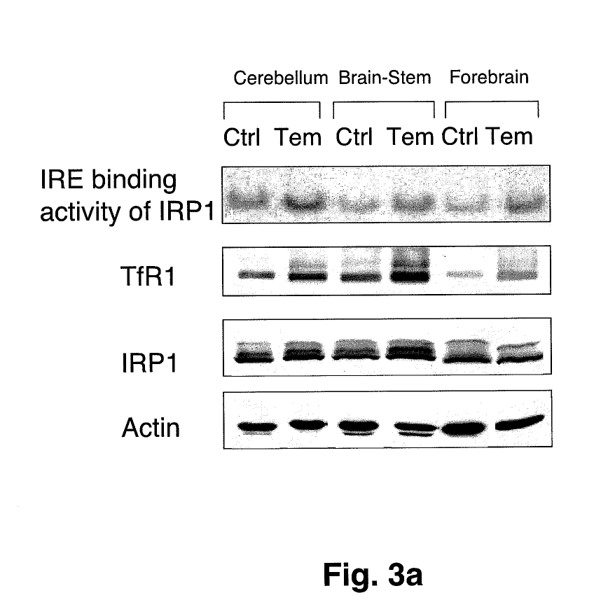

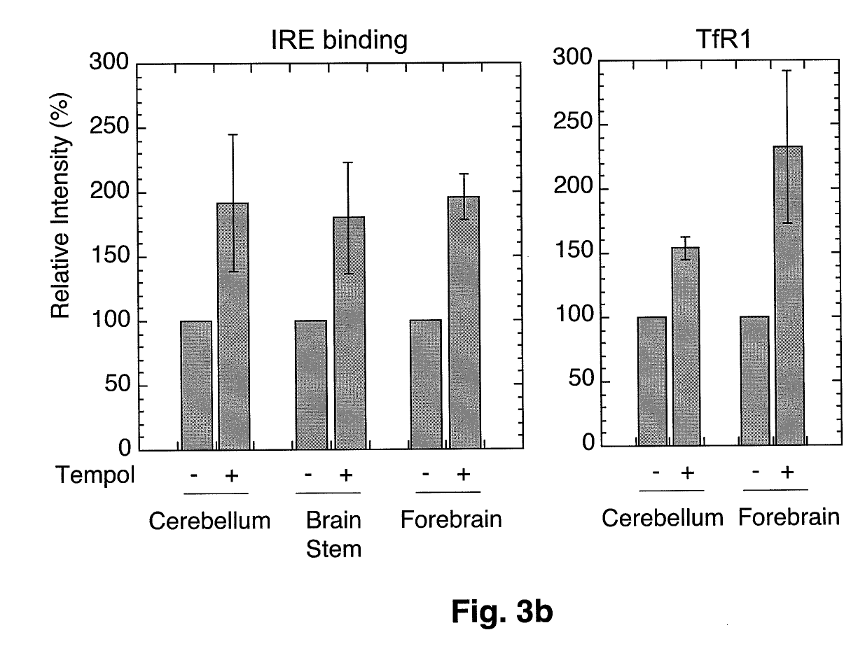

Lysates made from various brain regions of IRP2−/− mice that were maintained on a control diet or on a Tempo1-supplemented diet were analyzed for IRE binding activity of IRP1 and for TfR1, IRP1, and actin protein levels by gel-shift assay and Western blot. The results are depicted in FIG. 3a. FIG. 3b is a quantification of the results as intensity of the bands relative to the control, represented here as 100%. Quantification was performed with the IQMac (IRP1 activity) or NIH Image (protein levels) program. Error bars represent the standard deviation calculated from the results of two different sets of animals.

In lysates from the cerebellum, brain-stem, and forebrain, IRE-binding activity and TfR1 protein levels were markedly increased in Tempo1-supplemented mice as compared to the control mice. IRP1 and actin protein levels did not significantly change in these brain regions as a result of Tempo1 treatment. These results confirm that Tempo1 exerts a positive effect on IRE-binding activity of IRP1 in vivo.

### Example 4

The following example shows that treatment with a nitroxide radical reduces ferritin expression and ferric iron accumulation in the white matter of the brain.

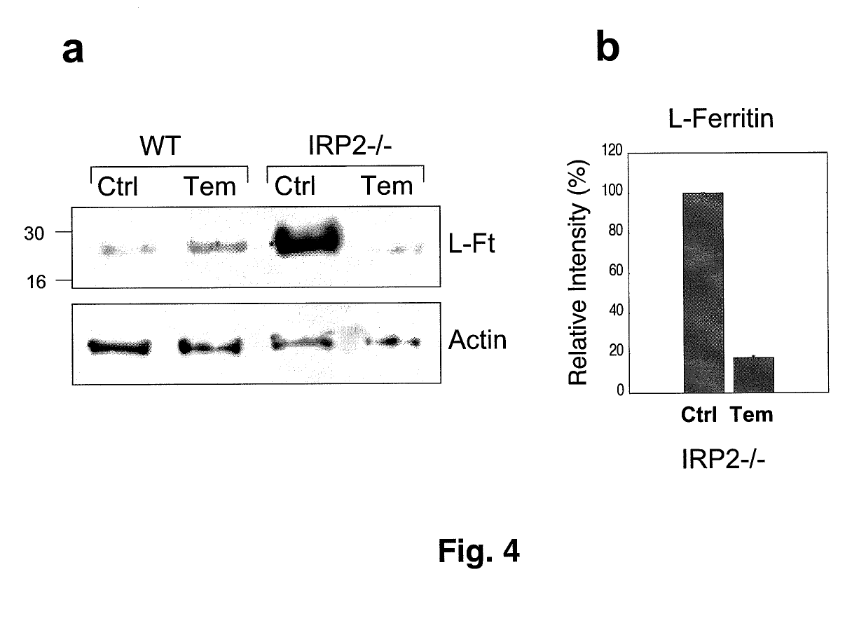

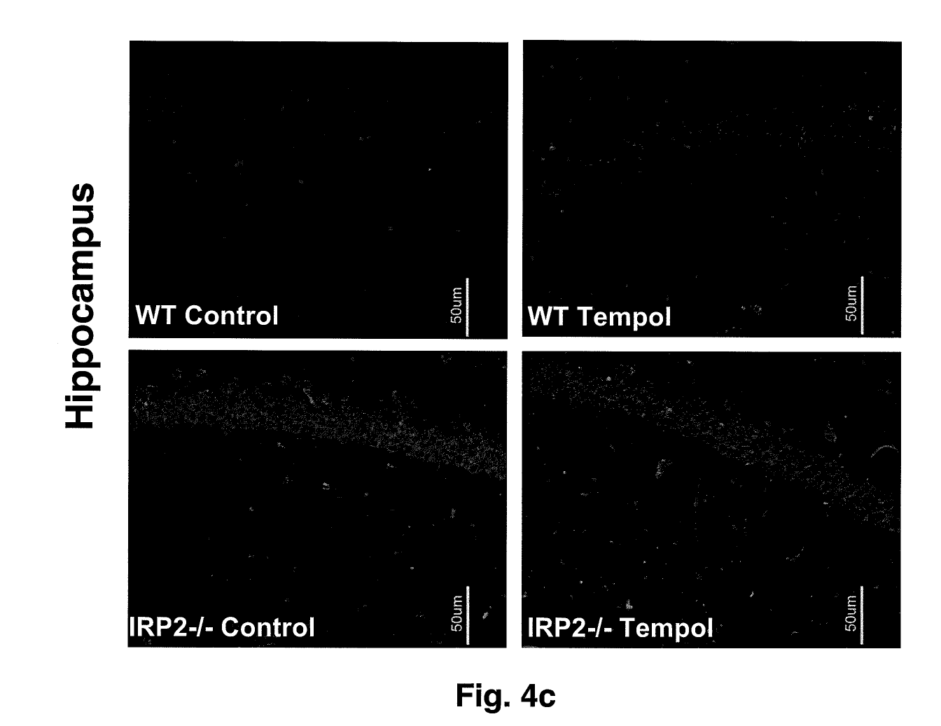

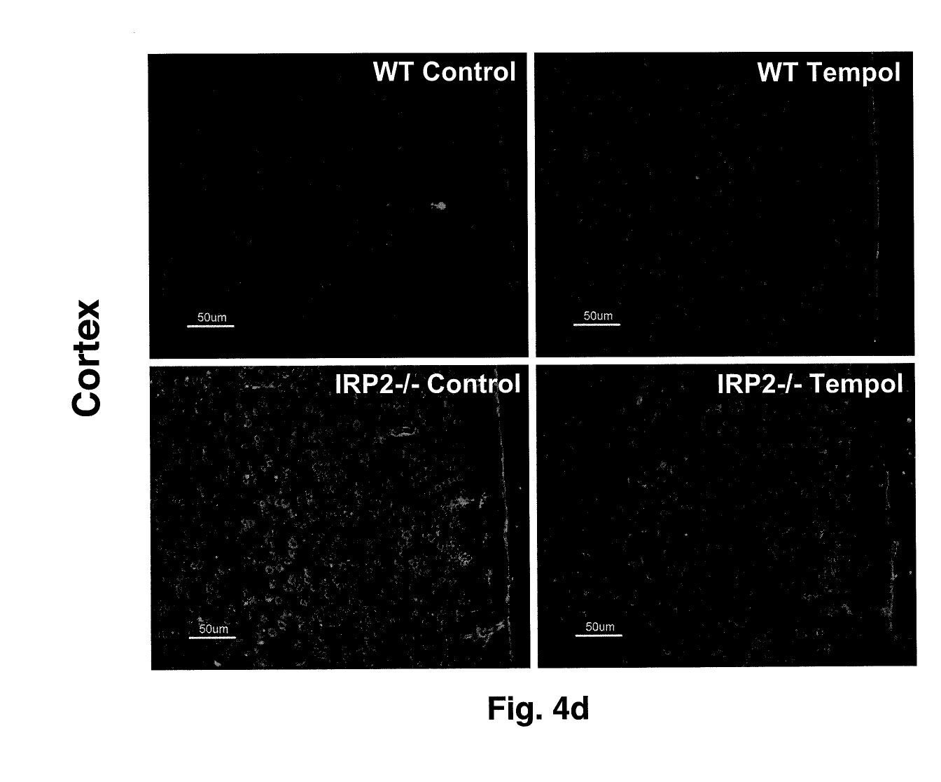

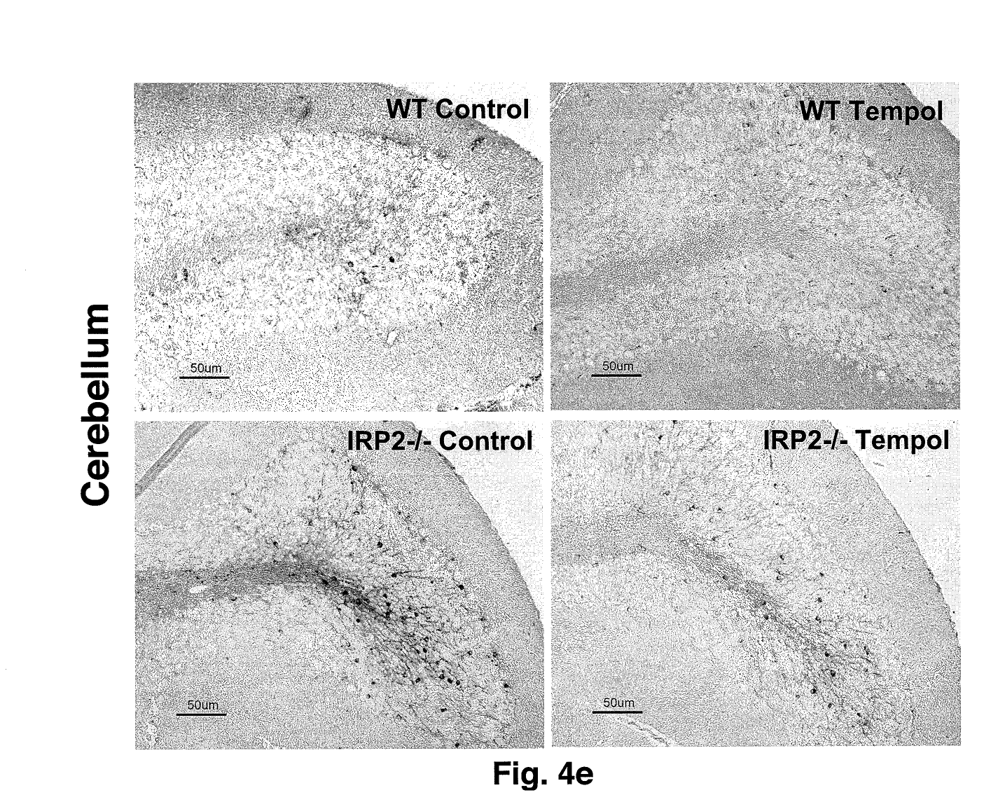

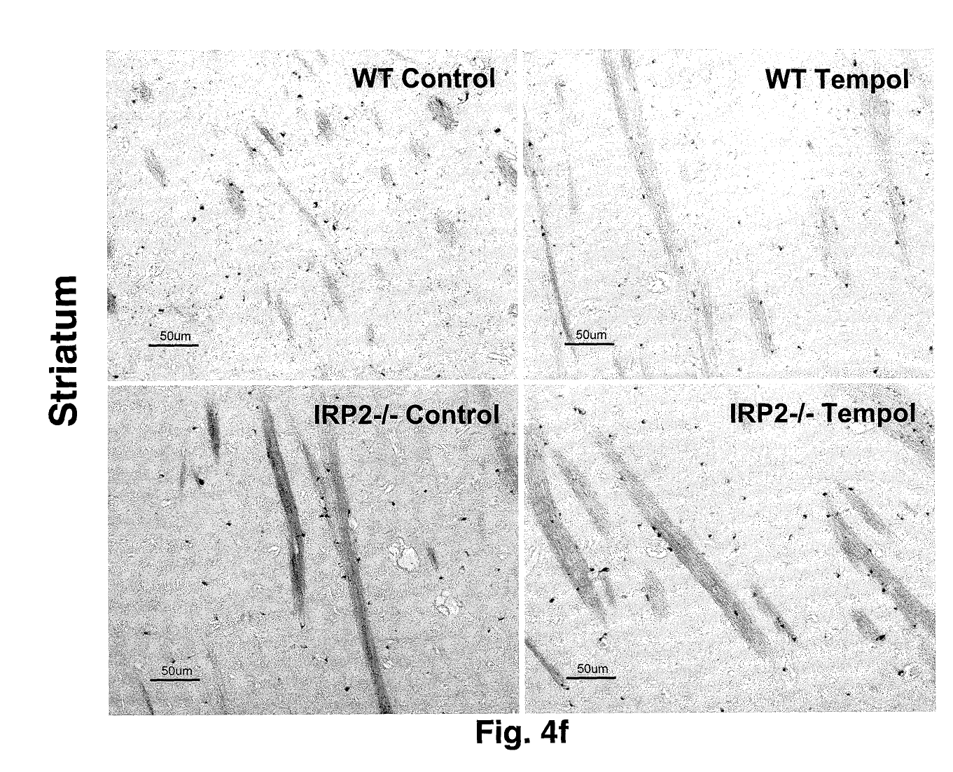

Cerebellar lysates from wild-type and IRP2−/− mice that were maintained on a control diet or on a Tempo1-supplemented diet were analyzed for ferritin and actin expression. FIG. 4a shows the results of the Western blot analysis. FIG. 4b shows the ferritin level as a measure of relative intensity as compared to the control, represented as 100%. Quantification was performed with the NIH Image program. Error bars represent the standard deviation calculated from the results of three different sets of animals. As the results show, ferritin levels were elevated in IRP2−/− mice on the control diet as compared to wild type mice. Treatment with tempol reduced ferritin levels in the IRP2−/− mice, consistent with the observed increase of IRE-binding activity induced by Tempo1.

Immunohistochemical studies were performed to analyze ferritin expression in the hippocampus and cortexregions of the mouse brains. The results are presented in FIGS. 4c and 4d. As the results show, ferritin expression was markedly increased in the hippocampus and cortex of the IRP2−/− animals (lower left panel) as compared to wild-type animals. The overexpression of ferritin decreased with Tempo1 treatment.

Cerebellar folia and striatum of wild type and IRP2−/− mice that were maintained on a control diet or on a Tempo1-supplemented diet were analyzed for ferric iron accumulation using Perls' DAB stain. The results are presented in FIGS. 4e and 4f. As shown in the figures, ferric iron staining increased in the tested regions of the IRP2−/− animals on control diet compared to the wild-type controls, indicated ferric iron sequestration in oligodendrocytes and in the cerebellar white matter tracts of IRP2−/− animals. The staining was decreased in IRP2−/− animals on the Tempo1 diet, indicated reduced iron sequestration.

These results show that stable nitroxide radicals can reduce ferritin expression and ferric iron accumulation in the brain.

### Example 5

The following example shows that a nitroxide radical recruits IRE-binding activity of IRP1 via disassembly of the iron-sulfur cluster of cytolic aconitase to generate an IRE-binding form of IRP1.

IRP1 is a bifunctional protein that alternates between two forms: in iron-replete cells, IRP1 contains a cubane iron-sulfur cluster and functions as a cytosolic aconitase that interconverts citrate and isocitrate, whereas upon loss of its redox-sensitive ironsulfur cluster, IRP1 undergoes a significant conformational change that enables it to bind to IREs. In animal tissues, most IRP1 contains an intact iron-sulfur cluster and functions mainly as an active aconitase.

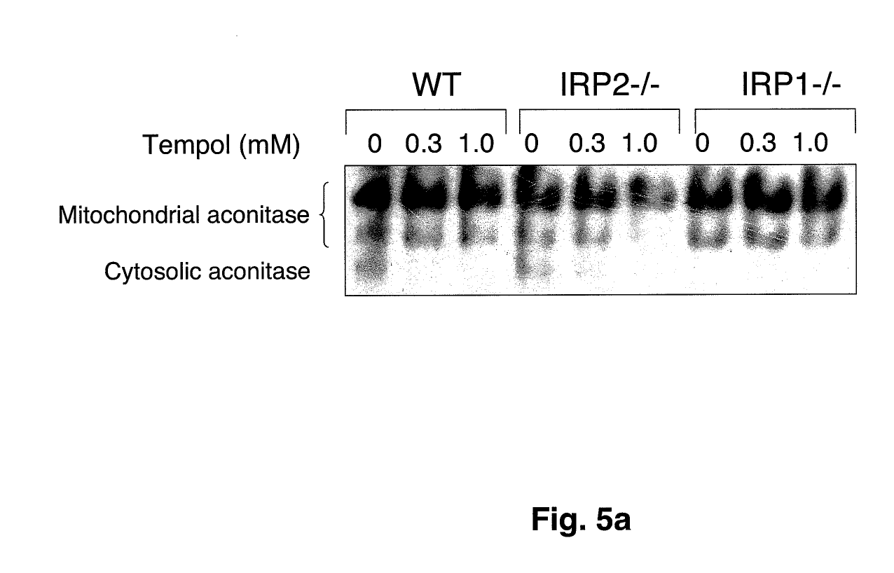

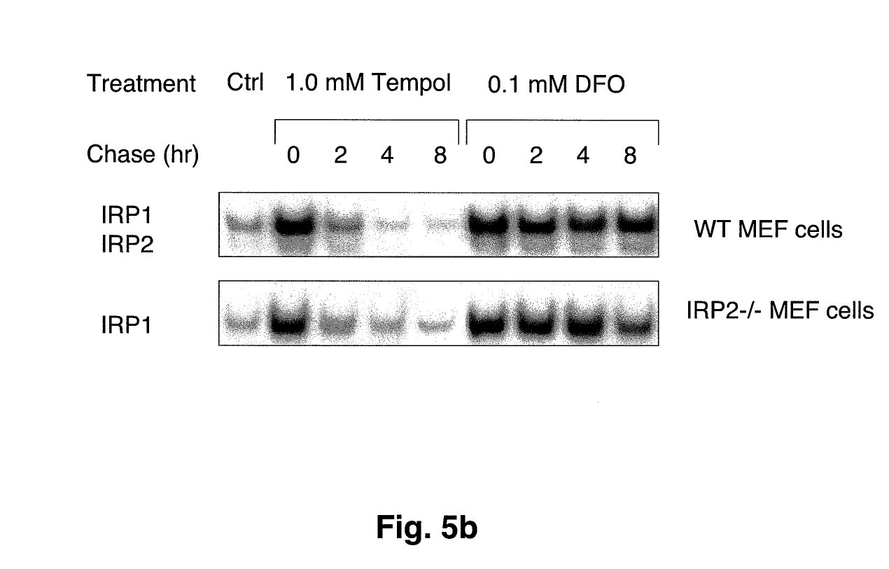

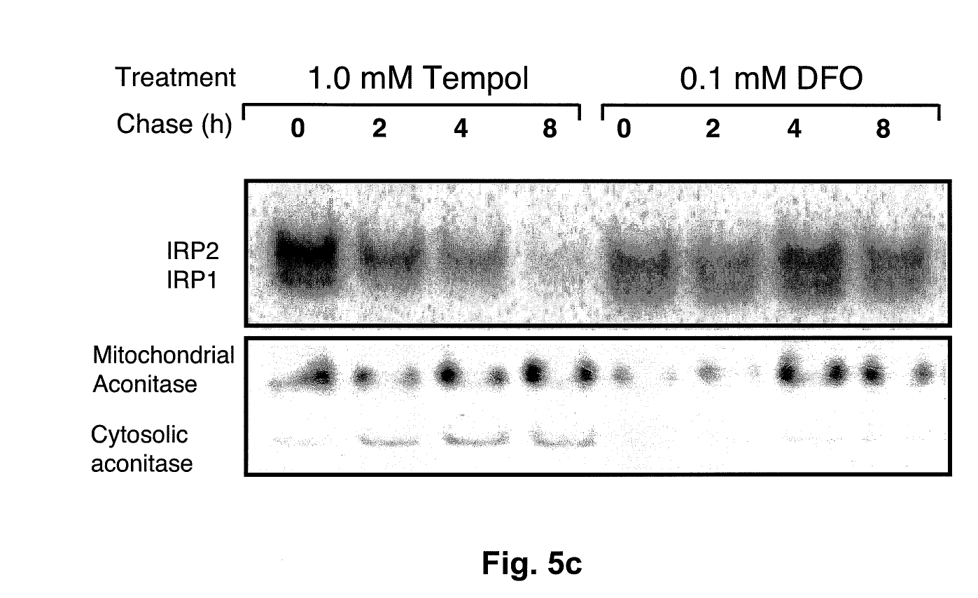

To confirm that nitroxide radicals activate IRP1 binding by disassembly of the iron-sulfur cluster, we performed in-gel aconitase assays to determine whether Tempo1 decreased the activity of cytosolic aconitase. FIG. 5a shows the cytosolic aconitase activity in mouse embryonic fibroblast (MEF) lysates. The band that represented the cytosolic aconitase was readily identified by its absence in lysates from IRP1−/− cells. Notably, Tempo1 treatment diminished the activity of cytosolic aconitase in lysates from WT and IRP2−/− embryonic fibroblasts. This is consistent with other observations in the art that small redox molecules, such as nitric oxide, O2⁻ and ascorbate, can react with the iron-sulfur cluster of the cytosolic aconitase, which leads to loss of the iron-sulfur cluster and conversion to the IRE binding form of IRP1. Tong et al., Cell Metab 3, 199-210 (2006); Bouton et al., Sci STKE 2003, pe 17 (2003).

To determine whether the effect of Tempo1 on IRP1 activity was kinetically consistent with oxidative disassembly of the iron sulfur cluster, the effect of Tempo1 on aconitase activity was compared to that of the iron chelator, deferiprone (DFO), which activates IRE-binding activity of IRP1 by limiting de novo synthesis and repair of the iron sulfur cluster in IRP1. Mouse embryonic fibroblasts from wild type and IRP2−/− mice were treated with Tempo1 or iron-cheltor deferiprone (DFO) for 16 hours, after which time they were switched to fresh unsupplemented media and subsequently assayed at various time points by gel-shift and aconitase gel assays. Recovery of aconitase activity was assessed in wild type and IRP2−/− cells. The results are presented in FIGS. 5b and 5c.

Both Tempo1 and DFO treatments activated the IRE-binding activity of IRP 1. However, the activation of IRE-binding activity and loss of cytosolic aconitase activity induced by Tempo1 was readily reversed within 2 hours after removal of Tempo1, whereas little recovery of cytosolic aconitase activity was discernible even when activity was assessed 8 hours after removal of DFO. The difference in the rate of iron sulfur cluster recovery after treatment with Tempo1 compared to DFO indicated that Tempo1 and DFO activated IRP1 by distinct mechanisms. The results suggest that the iron sulfur cluster of IRP1 was disassembled by Tempo1, but was readily rebuilt when the cluster destabilizing reagent was removed, whereas recovery of cytosolic aconitase activity after DFO treatment was limited by depletion of intracellular iron and reduced expression of iron-sulfur assembly proteins. In addition, treatment does not substantially affect mitochondrial aconitase activity, and was therefore relatively benign.

### Example 6

The following example shows that nitroxide radicals exert a direct effect on the iron sulfur cluster of IRP 1.

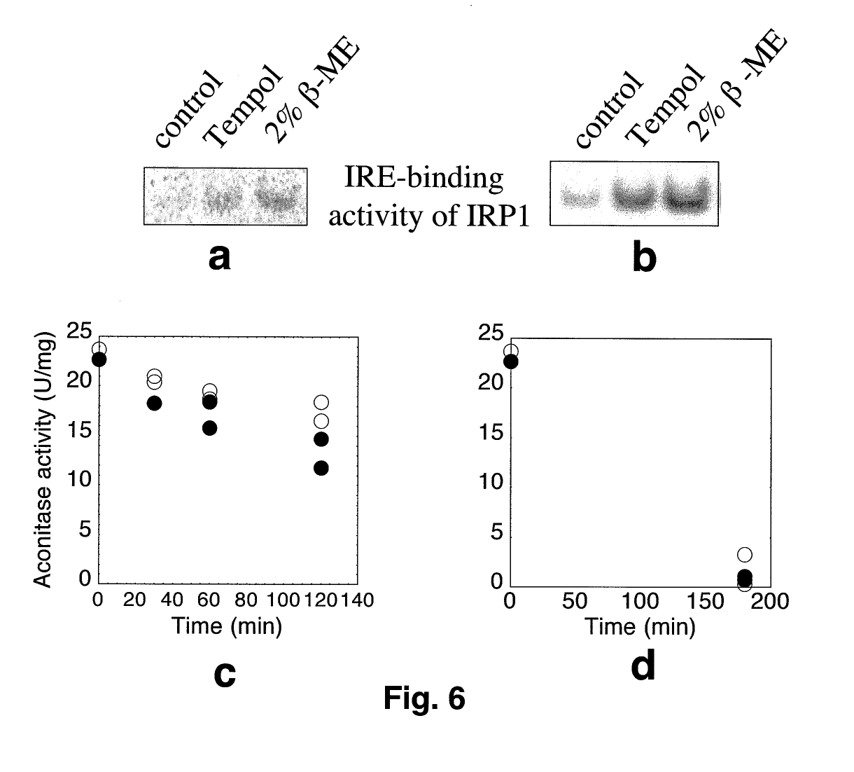

To assess whether the effect of Tempo1 on the iron-sulfur cluster status of IRP1 represents a direct chemical disassembly process, we treated purified holo-IRP1 (containing an intact [4Fe-4S] cluster) with Tempo1, using time and temperature conditions similar to those that have been used in the art to demonstrate disassembly of the iron-sulfur cluster of IRP1 by nitric oxide. Soum et al., J. Biol Inorg Chem, 8, 226-232 (2003). IRE-binding activity of purified holo-IRP1 (11 ng) was assessed by an IRE gel shift assay. Purified protein (11 ng) was incubated with 0.1% β-mercaptoethanol for 2 h at room temperature without (lane 1) or with (lane 2) 1 mM tempol. Sample in lane 3 was treated with 2% β-mercaptoethanol for 2 min after 2 h incubation without tempol. The results are presented in FIGS. 6a and 6b. Aconitase activity was measured by a coupled solution assay following the method of Fillebeen et al., Biochem J, 388, 143-150 (2005) using cis-aconitate as the substrate demonstrated comparable losses of aconitase activity over time in control and Tempo1 treated samples at room temperature and at 37° C. for 3 h. The results are presented in FIGS. 6d and 6e.

Treatment with Tempo1 increased IRE binding activity of IRP1, consistent with complete disruption of [4Fe-4S] cluster. IRE binding activity was much higher for IRP1 treated with Tempo1 for 2 h at room temperature, and for IRP1 treated with Tempo1 for 3 h at 37° C. as compared to IRP1 exposed to room air alone. FIG. 6b shows IRE-binding activity was almost completely recruited by treatment of holo-IRP1 by 1.0 mM Tempo1, for 3 h at 37° C. However, addition of Tempo1 to purified protein did not enhance the loss of aconitase activity that occurred with exposure to room air for up to 2 h or for 3 h at 37° C. These results showed that Tempo1 enhances disassembly of the cluster in assays using purified protein, whereas exposure to oxygen alone promotes fonnation of an identifiable intermediate that lacks aconitase activity, but retains remnants of a cluster that preclude IRE-binding.

These assays on purified protein demonstrated that Tempo1 directly and fully disassembles the iron-sulfur cluster of IRP1, and support the conclusion that Tempo1 activates IRE binding activity of IRP1 in cells and animals by directly destabilizing the iron-sulfur cluster of IRP1. The partially degraded form of the iron sulfur cluster might be repaired in cells and animals, but not in purified protein samples, which explains the results of in-vivo and in-vitro aconitase activity measurements after Tempo1 treatment.

### Example 7

The following example shows that nitroxide radicals exert a therapeutic effect through recruitment of IRP1 binding activity.

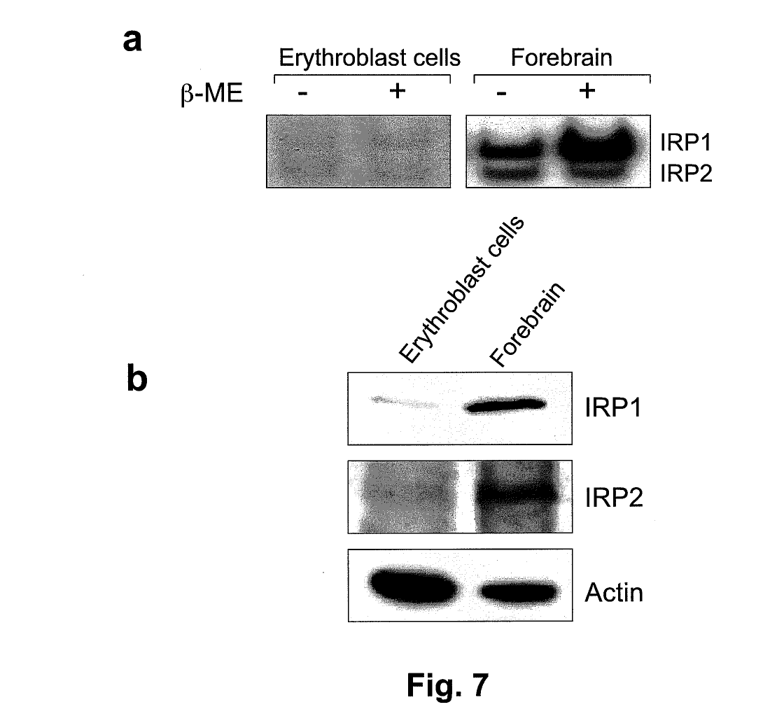

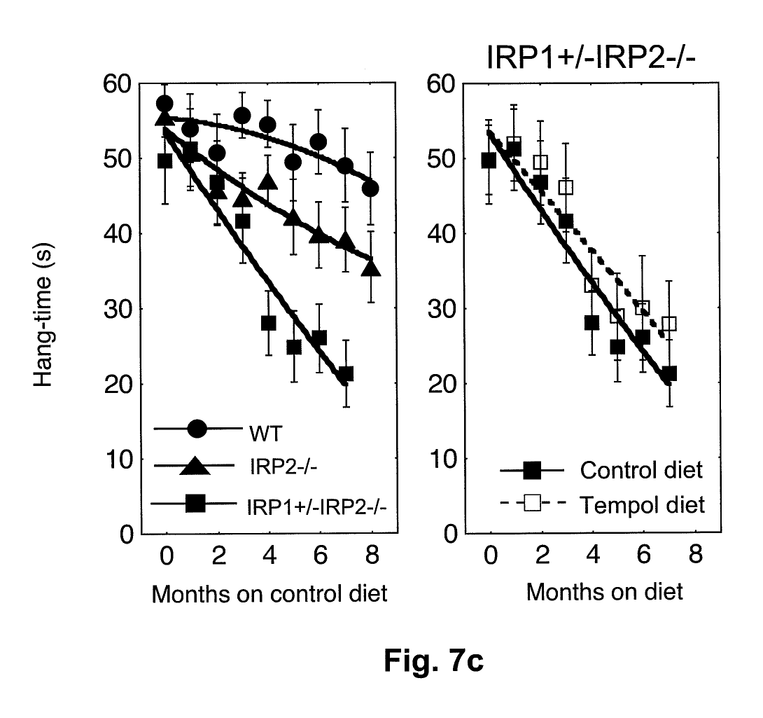

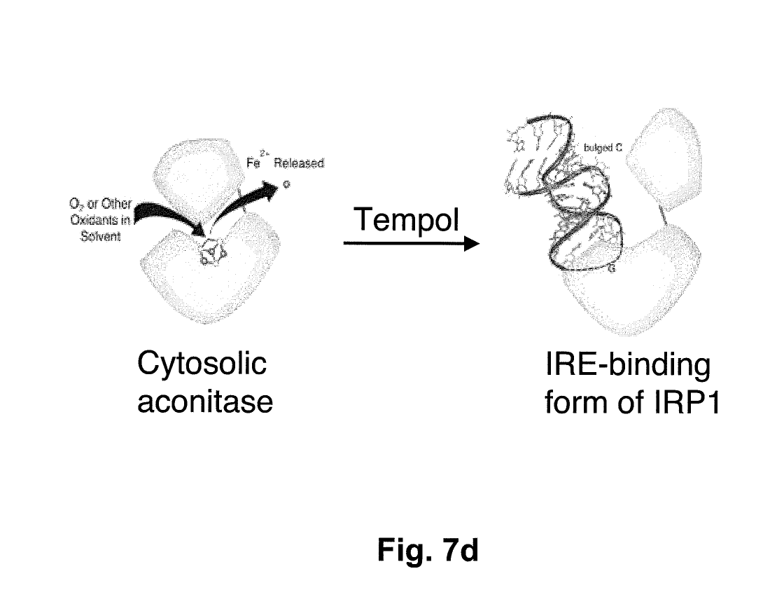

Erythroblasts were isolated from wild type mice to assess the relative activities of IRP1 and IRP2 and to determine whether IRP1 could be recruited to the IRE binding form from a latent pool of IRP1 in erythroblasts. Gel-shift studies indicated that IRP1 and IRP2 equally contributed to IRE-binding activity in erythroblasts (FIG. 7a). However, IRP1 levels were markedly decreased in erythroblasts compared to brain (FIG. 7b). Also, treatment of erythroblasts with high concentrations of β-mercaptoethanol, which converts IRP1 from the cytosolic aconitase form to the IRE binding form, did not activate additional IRE binding activity of IRP1 in erythroblasts. In contrast, significant increases of IRE binding activity were recruited from brain lysates using β-mercaptoethanol treatment (FIG. 7a), indicating that developing red cells lack a significant amount of IRP1 in the cytosolic aconitase form that can be converted to the IRE-binding form by treatment with Tempo1 or other iron-sulfur cluster destabilizing reagents.

These results explain why Tempo1 treatment of IRP2−/− mice prevented neurodegeneration, but did not lead to an improvement in the mild anemia of IRP2−/− mice, even though the mild iron-insufficiency anemia of IRP2−/− animals may be largely attributable to decreased expression of TfR1 in erythroblasts and decreased bone marrow iron stores. The mild anemia of IRP2−/− mice (hematocrit was 46±5 compared to 52±2 for WT, p=0.022) did not improve in animals treated with Tempo1, remaining at 46±4 after 8 months of Tempo1 diet. Tempo1 did not correct the anemia of IRP2−/− mice because very little IRE-binding activity could be recruited in erythroblasts.

Moreover, Tempo1 treatment protected the IRP2−/− animals from neurodegeneration, but did not significantly (p=0.559) prevent disease progression in the IRP1+/− IRP2−/− mice (FIG. 7c). Hang-test results of WT, IRP2−/− and IRP1+/− IRP2−/− mice that indicated IRP1+/− IRP2−/− animals were more symptomatic than IRP2−/− animals. However, Tempo1 treatment apparently did not fully protect IRP1+/− IRP2−/− animals significantly (p=0.559) from progression of neurodegenerative symptoms. Error bars represent standard error of the mean. The curves were drawn by using the polynomial curve fit of the KaleidaGraph program. Hang-test curves of WT and IRP2−/− mice shown in FIG. 1 are re-displayed here for comparison to IRP1+/− IRP2−/− animals. These results suggest that the loss of one IRP1 allele markedly reduced the amount of IRP1 in the IRP1+/− IRP2−/− mice that could be recruited to bind IREs.

Taken together, these results further support the conclusion that restoration of normal iron homeostasis by Tempo1 treatment depends upon conversion of sufficient amounts of IRP1 to the IRE-binding form.

All references, including publications, patent applications, and patents, cited herein are hereby incorporated by reference to the same extent as if each reference were individually and specifically indicated to be incorporated by reference and were set forth in its entirety herein.The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating or preventing neurodegeneration in a mammal afflicted with a neurodegenerative disease comprising administering to the mammal an amount of a stable nitroxide radical sufficient to treat or prevent neurodegeneration.

2. A method of increasing the amount of bioavailable iron in the central nervous system (CNS) of a mammal with a CNS iron deficiency comprising administering to the mammal a stable nitroxide radical in an amount sufficient to increase the amount of bioavailable iron in the central nervous system of the mammal.

3. A method of activating Iron Regulatory Protein 1 (IRP1) or increasing Transferrin Receptor 1 (TfR1) expression in a mammal comprising administering to the mammal a stable nitroxide radical in an amount sufficient to activate IRP1 or increase TfR1 expression in the mammal.

4. (canceled)

5. The method of claim 1, wherein the stable nitroxide radical is Tempo1 or a hydroxylamine analogue thereof.

6. The method of any of claim 1 wherein the stable nitroxide radical is Tempo1-H.

7. The method of any of claim 1, wherein the mammal is deficient in Iron Regulatory Protein 2 (IRP2) function.

8. The method of any of claim 1, wherein the mammal under-expresses TfR1.

9. The method of any of claim 1, wherein the mammal is afflicted with a neurodegenerative disease characterized by abnormal accumulations of ferric iron in the CNS.

10. The method of any of claim 1, wherein the mammal is afflicted with Parkinson's Disease, Alzheimer's Disease, Hallevorden-Spatz, aceruloplasminemia, refractory anemia, erythropoietic protoporphyria, or adult-onset neurodegeneration.

11. The method of any of claim 1, wherein the mammal is a human.

12. The method of any of claim 1, further comprising administering to the mammal an iron supplement or high iron diet.
